(19) United States
(12) Patent Application Publication
(10) Pub. No.: US 2008/0194540 A1
(43) Pub. Date: Aug. 14, 2008

(54) Use of a Tricyclic Antidepressant Drug For Promoting Endocytosis

(75) Inventors: Birgit Neukamm, Berlin (DE); Christine Lang, Berlin (DE); Reinhard Gessner, Berlin (DE)

Correspondence Address: MAYER & WILLIAMS PC, 251 NORTH AVENUE WEST, 2ND FLOOR, WESTFIELD, NJ, 07090, US

(73) Assignee: RINA NETZWERK RNA-TECHNOLOGIEN GMBH, BERLIN (DE)

(21) Appl. No.: 10/548,207

(22) PCT Filed: Feb. 17, 2004
(86) PCT No.: PCT/DE04/00321

§ 371 Date: Sep. 26, 2006

(30) Foreign Application Priority Data
Mar. 6, 2003 (DE) 103 10 196.9

(51) Int. Cl. A61K 31/553; C07D 267/16; C12N 15/01; C12N 5/00

(52) U.S. Cl. 514/211.11; 540/547; 435/441; 435/325

(57) ABSTRACT

The invention teaches the use of a tricyclic compound for promoting the endocytotic uptake of macromolecular active ingredients.

### FIELD OF THE INVENTION

The invention relates to the use of a substance for preparing a pharmaceutical composition for promoting endocytosis of an active ingredient, in particular a construct comprising a nucleic acid or composed thereof, for instance for a gene therapy or the transfection of mammal cells. The invention further relates to methods for promoting endocytosis and to cells, which are transformed with such a method.

### BACKGROUND OF THE INVENTION

In various therapeutic approaches, it is necessary to use active ingredients having high molecular weights. Some examples are briefly described in the following.

Gene therapy is a promising method for curing a plurality of diseases, which are caused by a germline or somatic gene defect, such as hereditary diseases and cancer (Crit Rev Ther Drug Carrier Syst 1999; 16(2):147-207). In gene therapy, nucleic acids are introduced into the target cell, and are intended to directly clear the defect there. In the last decades, several methods for introducing nucleic acids into target cells were developed. Thereto belongs the introduction of nucleic acids into the cell by means of viral vectors, the lipofection and the directed uptake by means of receptor-mediated endocytosis, such as the transferrinfection. The drawbacks of viral systems compared to the remaining systems became increasingly obvious in the last years, such that the future of the gene therapy will probably require the use of other systems. This is a method, which makes use of the natural endocytosis mechanisms of the cell. The transferrinfection is based on the natural transferrin-transferrin receptor endocytosis system guaranteeing the iron supply in proliferating, differentiating and hemoglobin-synthesizing human cells (Theil, E. C., Aisen, P. (1987) The storage and transport of iron in animal cells. Iron transport in microbes, plants, animals, pp. 491-520, Winkelmann, G., van der Helm, D. and Neilands, J. B. (editors), VCH, Weinheim). In the transferrinfection system, the naturally occurring transferrin-transferrin receptor endocytosis system was modified by coupling the DNA to the ligand transferrin (E. Wagner et al., 1991, Bioconjugate Chem. 2:226-231). The ligand specifically binds to the cell surface receptor and is taken up by endocytosis. In order to neutralize the negative charge of the DNA, to condensate the DNA and to make it thus accessible to an uptake, polycations such as polyethylenimine or polylysine are used. It could be shown, further, that condensed DNA not coupled to a ligand is endocytotically taken up (W. T. Godbey et al., 1996, PNAS 96:5177-5181), and this system is described as polyfection. For a gene therapeutic use of the systems developed up to now, for the target-directed introduction of nucleic acids into eukaryotic cells by endocytosis, however, the uptake rate, stabilization by DNA against degradation in the cell's own compartments and transfer efficiency to the active position (cytosol or nucleus) is insufficient (Mahato R I (1999) Non-viral peptide-based approaches to gene delivery. J Drug Target. 7(4):249-68).

Besides gene therapy, there are further treatment approaches and uses of in most cases macromolecular nucleic acids. A first approach is the introduction of directly translatable RNA into a target cell, and then an active ingredient coded by the RNA is expressed by the cell's own mechanisms. By means of anti-sense RNA, RNA aptamers, siRNA and ribozymes, processes naturally taking place in a cell because of malfunctions, mutations etc. can be modulated on an RNA level. In this context, too, the use and modulation or promotion of natural endocytosis processes is necessary, in order that the nucleic acid is introduced in a sufficiently large amount into the cell.

Corresponding considerations apply to other low and/or macro-molecular molecules in therapeutic applications, which can only difficultly or not at all overcome the membrane barrier or be taken up in the cell in the complex with proteins, nucleic acids or synthetic macromolecules by means of endocytosis. Thereto belong in particular all drugs that are present in an ionized form in the plasma and for that a suitable cellular transport system does not exist. Further it is desirable to have available a method for taking-up lipophilic pharmaceuticals, which have a high protein binding and can be taken up in the target cell by endocytosis after in vitro binding to a suitable carrier protein or another suitable macro-molecule.

Further, in vitro transfected cells can in turn be used for therapeutic purposes. For instance, tumor cells can be taken from a patient, cultivated in vitro and specifically modified by gene technology, for instance by means of an expression cassette for a gene stimulating an immune reaction against the tumor cells. These modified cells can then be administered again to the patient and can then cause immune reactions in the patient, said immune reactions being directed against the not modified tumor cells of the patient. Thus, a patient-specific and tumor-specific tumor vaccine is obtained.

When using oncolytic viruses, special viruses are administered to an organism diseased with cancer. These viruses have the property, in principle, to infect all cells, however to multiply exclusively in tumor cells, and to initiate cell lysis in these tumor cells. In healthy cells, these viruses are however resting. It is for instance known to use modified herpes simplex viruses (HSV) for treating brain tumors.

Particularly in therapeutic applications, there is thus the basic problem to transport the active ingredient from the extracellular space into the cell and from the endocytotic compartments into the cytosol, i.e. to cause endocytosis, or to amplify a naturally occurring endocytosis, in order that less active ingredient is required or the transfection rates are sufficient. The term endocytosis generally designates, in this specification, the uptake of extracellular, corpuscular or dissolved, in most cases macro-molecular material by a cell. Macro-molecular means compounds having a molecular weight above 300 da, preferably above 500 da, most preferably above 1,000 da. To the macro-molecules than can naturally be taken up belong for instance antibodies, enzymes, antigen-antibody complexes, lipoproteins, LDL, transferrin and nucleic acids. Corpuscular material that can naturally be taken up comprises for instance viruses, bacteria and protozoa. For instance for therapeutic measures, but also when using directly translatable RNA molecules or nucleic acids acting as antisense-RNA, ribozymes, siRNA or RNA aptamers (including non-natural derivatives such as PNA), the macro-molecular nucleic acids, at least as a first step, need to taken up in the cytosol, if applicable also further transported into the nucleus. Methods and auxiliary agents such as the low-molecular active ingredients favored up to now in the pharmaceutical industry are not suitable for this. Rather, the natural endocytosis mechanisms need to be used, and the transport rate from the cell membrane to the effective position needs to be increased to an acceptable level by suitable auxiliary substances.

Such auxiliary substances are substances, which act on one or several steps of the endocytosis and increase the transport rate or modulate the latter. It is known to use quinoline ring structures, such as chloroquine, as a substance promoting the endocytosis. For this purpose, as an example only, reference is made to the document B. Neukamm et al., Biochimica et Biophysica Acta, 1572:67-76 (2002), as well as to the quotations therein. Further, it is known to use phenothiazine, such as chlorpromazine, for promoting the endocytosis.

From other applications, tricyclic substances are known, for instance desipramine or amoxapine. These compounds are antidepressants.

### TECHNICAL OBJECT OF THE INVENTION

It is the technical object of the invention to provide means, which improve the endocytosis of macro-molecular active ingredients.

### BASICS OF THE INVENTION AND EMBODIMENTS

For achieving this technical object, the invention teaches the use of a substance according to formula I or II or of several different such substances

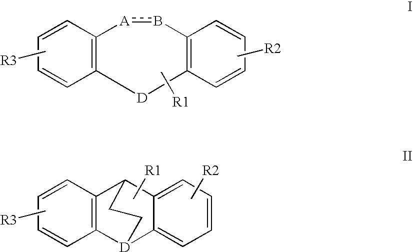

wherein A, B and D are identical or different and are each a C, N, S or O atom, wherein . . . is a single or double bond, wherein free valences are bound with —H, wherein R1 is present one, two or three times and —H, —C1-15-alkyl (branched, linear or cyclic) or —C1-15-alkyl (branched, linear or cyclic) is substituted with one or several C atoms by O, S or N, wherein R2 and R3 are identical or different, each and independently from each other are present one, two, three or four times and are —H or -Hal, wherein -Hal=—F, —Cl or —Br, for preparing a pharmaceutical composition for promoting the endocytosis of active ingredients. In the case of R1 with a substituted C atom or several substituted C atoms, the O, S or N atom is counted as a C atom for the term C1-15-alkyl. For instance, a residue R1-CH2-CH2-CH2-NH—CH3 would be, in this terminology, C5-alkyl, with one C atom being substituted by one N atom.

A substance is promoting the endocytosis, if the substance causes in a transfection experiment, as described in the examples, higher transfection rates or efficiencies in comparison to the same experiment, however only using the buffer (negative control). The degree of promotion can be determined and compared in an identical manner, if the quantity to be evaluated is quantitatively determined, absolute, relative to a negative control or relative to a defined reference substance promoting the endocytosis.

The invention is based on the surprising finding that so-called tricyclic antidepressants have an effect promoting the endocytosis.

In detail, the substance may comprise the following structural features. R1 may be a secondary or tertiary amine. Preferred examples for R1 are:

—CH₂—CH₂—CH₂—NH—CH₃

—CH₂—CH₂—CH₂—N(CH₃)₂

—CH₂—CH(CH₃)—CH₂—N(CH₃)₂

═CH—CH₂—CH₂—NHCH₃

═CH—CH₂—CH₂—N(CH₃)₂

1-piperazinyl

R1 may in particular be present once and be bound to D, and in the case of formula II, D may be a C atom . . . may be a single bond. A and B may be C atoms, and D a C or N atom. Alternatively, A may be a C atom, B an O atom and D a C atom. R2 and R3 may in particular be —H. A may be an N atom, B a C atom and D an O atom, with . . . being a double bond. R2 may be present once and be -Hal, and wherein R1 is —H.

Examples for suitable substances are: desipramine, imipramine, trimipramine, amitriptyline, nortriptyline, doxepin, amoxapine, maprotyline and other tricyclic antidepressants.

A preferred embodiment of the invention having an independent importance is characterized by that the pharmaceutical composition in addition comprises a quinoline ring compound and/or a phenothiazine compound, optionally selected from the group consisting of “chloroquine, chlorpromazine, primazine, quinine, biquinoline, phenothiazine and chlorpromazine”. A variant of this embodiment generally comprises the use of a mixture of at least two different substances selected from the group consisting of “quinoline ring compounds and/or phenothiazine compounds promoting the endocytosis, a substance promoting the endocytosis according to one of claims 1 to 9” for preparing a pharmaceutical composition comprising an active ingredient different from the substances and having a molecular weight of larger than 300. These embodiments of the invention are based on the finding that such a combination of known substances promoting the endocytosis with new substances used according to the invention, but also exclusively of already known substances promoting the endocytosis, will lead to synergistic effects. Surprisingly, transfection rates are obtained, which are substantially higher than those that are obtained with the respective substances alone. These explanations apply in particular also with regard to the second variant for the aspects of the invention described below, too.

It is also possible to use so-called triple or multi-combinations with three or more different substances discussed above.

Alternatively or additionally to the combination with the group mentioned in the section above, the combination may also be made with an alkyl polyamine. Alkyl polyamines having molecular weights between 100 and 50,000 may be used. Preferably, the alkyl polyamines are linear. Further is preferred that the polyamines have one or two primary amine groups. However, branched alkyl polyamines, if applicable with more primary amine groups, may in principle also be employed.

The pharmaceutical composition may basically contain every active ingredient, the introduction of which into a cell is desirable. In an important field of use, the active ingredient contains a construct including a nucleic acid having a defined sequence or consisting thereof, in a mixture with the substances used according to the invention or in a separate administration unit thereof and intended for common use. In the latter case, the administration units may be administered at the same time or one after the other, in both possible orders.

A defined sequence is a selected and given known sequence. The construct may comprise several such sequences. In the latter case, the several sequences may also be different, in particular for instance be variants of a sequence, within which partial sequences are specifically randomized. Such a randomization may concern 1 to 10, preferably 1 to 4 nucleotides connected to each other or not connected to each other. Randomization means that a randomized nucleotide position may comprise an arbitrary one of the four different nucleotides.

The invention further teaches the use of a substance employed according to the invention or several different such substances, optionally in a mixture with a quinoline ring compound and/or a phenothiazine compound, optionally selected from the group consisting of “chloroquine, primazine, quinine, biquinoline, phenothiazine and chlorpromazine”, for promoting the endocytosis of an active ingredient, in particular of a construct comprising a nucleic acid having a defined sequence or consisting thereof, wherein the cell is incubated in vitro with the active ingredient and the substance.

The invention further teaches a method for transiently or stably transfecting a cell in vitro, wherein the cell is incubated with a construct comprising a nucleic acid having a defined sequence or consisting thereof, and a substance used according to the invention or several different such substances, optionally in a mixture with a quinoline ring compound and/or a phenothiazine compound, optionally selected from the group consisting of “chloroquine, primazine, quinine, biquinoline, phenothiazine and chlorpromazine”, wherein the substance causes the endocytotic uptake of the nucleic acid in the cell (endocytosis), and wherein the nucleic acid comes into the cell and develops its effects therein (e.g. transfects the cell). The construct may comprise a marker gene, or the incubation may be performed with a second construct including a nucleic acid coding for a marker gene or consisting thereof. This variant permits an easier control of the transfection by detection of the expression product of the marker gene (e.g. GFP, luciferase measurement).

Finally, the invention teaches a stably or transiently transfectable cell, which can be obtained by incubating a cell with a construct comprising a nucleic acid having a defined sequence or consisting thereof, and a substance used according to the invention or several different such substances, optionally in a mixture with a quinoline ring compound and/or a phenothiazine compound, optionally selected from the group consisting of “chloroquine, primazine, quinine, biquinoline, phenothiazine and chlorpromazine”, wherein the substance causes the endocytotic uptake of the construct, and wherein the nucleic acid transfects the cell.

The galenic preparation of a pharmaceutical composition according to the invention may be performed in a usual way. As counter-ions for ionic compounds and/or active ingredients can for instance be used Na⁺, K⁺, Li⁺ or cyclohexyl ammonium. In particular amines may be present as hydrochloride. Suitable solid or liquid galenic preparations forms are for instance granulates, powders, dragees, tablets, (micro) capsules, suppositories, syrups, juices, suspensions, emulsions, drops or injectable solutions (IV, IP, IM), and preparations with protracted release of active ingredient, for the production of which usual means are used, such as carrier substances, explosives, binding, coating, swelling, sliding or lubricating agents, tasting agents, sweeteners and solution mediators. As auxiliary substances are named here magnesium carbonate, titanium dioxide, lactose, mannite and other sugars, talcum, milk protein, gelatin, starch, cellulose and derivatives, animal and vegetable oils such as cod-liver oil, sunflower oil, peanut oil or sesame oil, polyethylene glycols and solvents, such as sterile water and mono or multi-valent alcohols, for instance glycerin. A pharmaceutical composition according to the invention can be produced by that at least one substance used according to the invention is mixed in a defined dose with a pharmaceutically suitable and physiologically well tolerated carrier and possibly further suitable active, additional or auxiliary substances, and is prepared in the desired form of administration.

The invention can be employed in the most various areas. Target cells, in vitro or in vivo, may in principle be all primary cells of an organism, for instance somatic pluripotent cells or T cells, and all cell lines to be applied for vitro experiments. For instance in the case of T cells, the substances used according to the invention are advantageous over chloroquine. Special applications are for instance the gene therapy, the generation of organisms, such as lab animals, for certain test purposes, however also in the transplantation medicine, and storage damages of the cells of the organs to be transplanted are prevented or repaired and/or the immune behavior of the organs can be modified such that rejection reactions in the body of the recipient do not take place.

In the following, the invention is explained in more detail with reference to embodiments and reference examples.

### EXAMPLE 1

### Active Ingredient Comprising a Nucleic Acid (Test Form)

For the purpose of measuring the substances according to the invention promoting the endocytosis, the plasmid described in the following was used as a model active ingredient. The plasmid pFL1 was used, as described in the document D. Botstein et al., Gene 8:17-24 (1979). pFL1 is a 2 μm circular plasmid for Saccharomyces cerevisiae (Sa) with a high copy number. It contains the Sa gene URA3 as a selective marker for ura3auxotrophic yeast strains and parts of the pBR322 E. coli plasmid for the replication and selection in E. coli. The plasmid was held in E. coli SF8 and purified with the Qiagen Plasmid Mega Kit (Qiagen, Hilden, Germany).

For luciferase cell culture assays, the luciferase gene was cloned with NotI (NEB) in pBlueskript SK (+) (Stratagene, Heidelberg, Germany), and that under the control of a CMV promoter and a SV40 poly-A signal. This plasmid was amplified in E. coli DH5α. The DNA was purified with the Qiagen Plasmid Maxi Kit.

### EXAMPLE 2

### Model Target Systems

### 2a: Yeast System.

A first cell system is a yeast system. The transfection protocol corresponded to that of the document B. Neukamm et al., Biochimica et Biophysica Acta 1572:67-76 (2002). For the measurements, a lab robot (Zinsser, Frankfurt, Germany) was used, and a pipetting protocol was prepared.

Yeast precultures were drawn from cultures of a −70° C. glycerol stock of the strain RPY10 (R. C. Piper et al., Eur J Cell Biol 65:305-318 (1995)) for 72 hours. In the main culture, the cells were drawn over night in YPD medium up to a cell density of 5 to 8·10⁷ cells/ml. 12·10⁹ cells were harvested and washed twice with the same volume of distilled water. The cells were made competent for the transfection by soaking in distilled water for 30 minutes at 4° C. The competent cells were harvested and resuspended in 10 ml 34% sucrose, adjusted with HCl to pH 4. The suspension was immediately transferred into a sterilized 40 ml glass tube (Zinsser Analytik, Frankfurt, Germany), which were then sealed by an aluminum foil. The tubes were then placed on a shaker on the platform of the lab robot and treated for 40 seconds at 300 rpm. The suspension was automatically distributed into the wells of a 96-well microtiter plate (80 μl each by using stainless steel tips).

The subsequent pipetting protocol was as follows. First, solutions of a substance according to the invention or of a reference substance in different concentrations were added. Exactly 5 minutes later, 10 μl of a solution or suspension of the pFL1 plasmid (0.15 μg/μl) of example 1 were automatically added to the mixture of cells and substance. Some wells were used for a negative control, and only the used solvents and the plasmid were added to the cell suspension, not however the substance. After termination of the pipetting protocol, the plate was covered and placed on the Desyre mixer (Zinsser Analytik). After vortexing for 1 minute at 1,300 rpm, an incubation was performed at 28° C. for 18-20 hours. After the incubation, 110 μl distilled water each were slowly added. Then the cells of each well were transferred to 6-well plates (PS Macroplate, Greiner), filled up with 4 ml WMIXura (Neukamm et al., see above). The wells of the microtiter plate were each washed with 50 μl distilled water, and the washing solution was pipetted on the solid surface of the WMIXura. The macroplates were left dry and incubated for 4 days at 28° C.

Colony forming units (cfu) were then counted in every well. The obtained cfu values were related to the cfu values of the negative control. This ratio is a measure for the transfection effectivity.

### 2b: Mammal Cells System.

HepG2 cells were drawn in Dulbecco's Modified Eagle Medium (DMEM, Gibco BRL, Eggenstein, Germany), supplemented with 10% FBS (fetal bovine serum, Gibco) and 1 μg/ml insulin. On day 1, the cells were removed by means of trypsination, washed and distributed on the wells of a 24-well tissue culture dish, namely 1.5·10⁷ cells per well.

Approx. 24 hours later the transfection tests were performed with the luciferase plasmid from example 1 with different substances at approx. 70 to 80% confluence. The supernatant was removed, and then a mixture (250 μl) containing medium, 12.5 μl dextran (10 mg/ml), 0.5 μl of the plasmid and the respective substance was added to every well. After 2.5 hours incubation at 37° C., the supernatant was removed, and 500 μl complete medium were added to each well. On day 5, the luciferase activity was measured by using the Dual Luciferase® Reporter Assay Kit (Promega, Mannheim, Germany). For this purpose, 48 hours after the transfection, the cell culture supernatants were removed, and the cells were solubilized after addition of 100 μl 1× lysis buffer per well (Promega) for 20 minutes at 20° C. on a slow shaker. 20 μl of the cell-free supernatant were transferred into a 96-well plate (Corning and Costar, Wiesbaden, Germany) and reacted with 100 μl luciferase assay buffer. The luciferase activity was measured with a luminometer (EG&G Berthold MicrolumatPlus).

### EXAMPLE 3

### Determination of the Optimum Concentrations

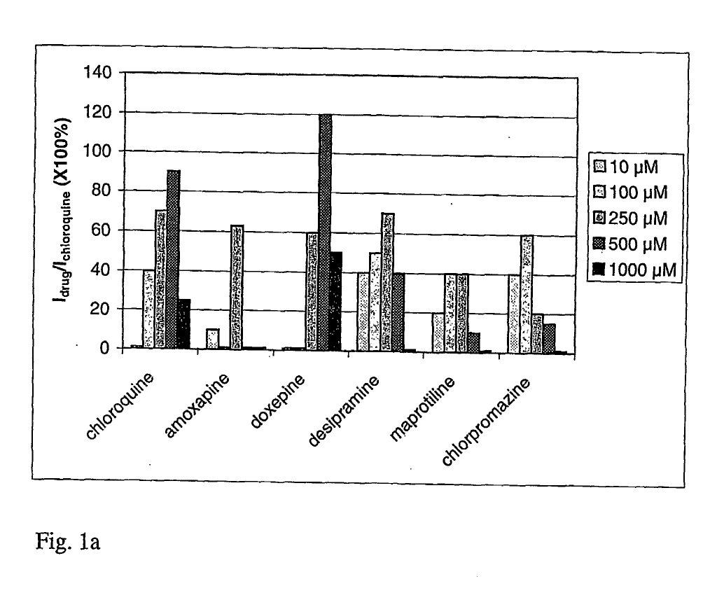

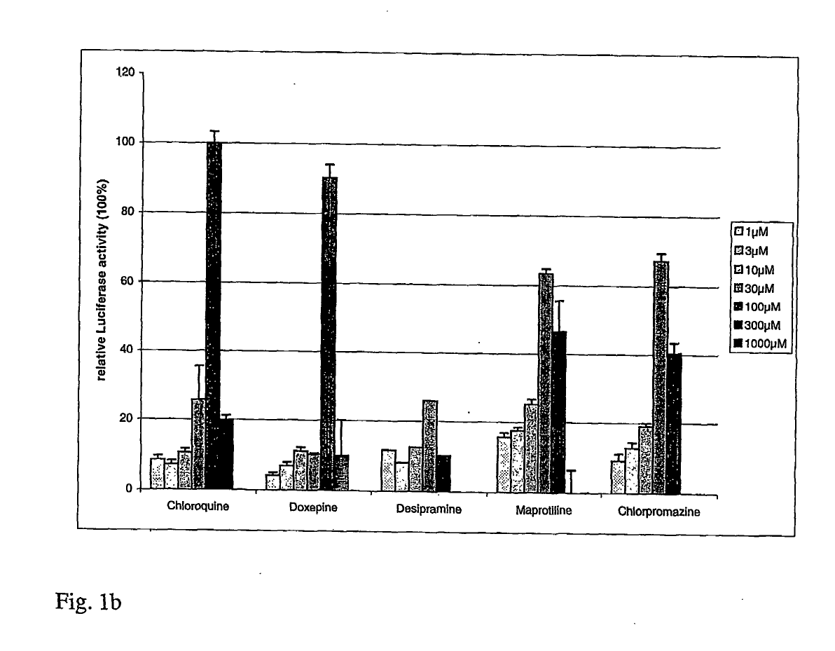

For each used substance, the optimum concentration was determined by means of concentration series. For this purpose, concentration series of the respective substances were used. For comparing, corresponding measurements were performed with chloroquine. The results are shown in FIGS. 1a (yeast cells) and 1b (mammal cells system). The ordinate values are standardized to chloroquine. It can be seen that in the case of the substances amoxapine, maprotyline and chlorpromazine, the optimum concentrations are smaller than in the case of chloroquine. In principle, with regard to side effects, as low concentrations as possible are desirable and advantageous. Only in the case of doxepine, the optimum concentration is similarly high as for chloroquine.

### EXAMPLE 4

### Mixtures of Substances

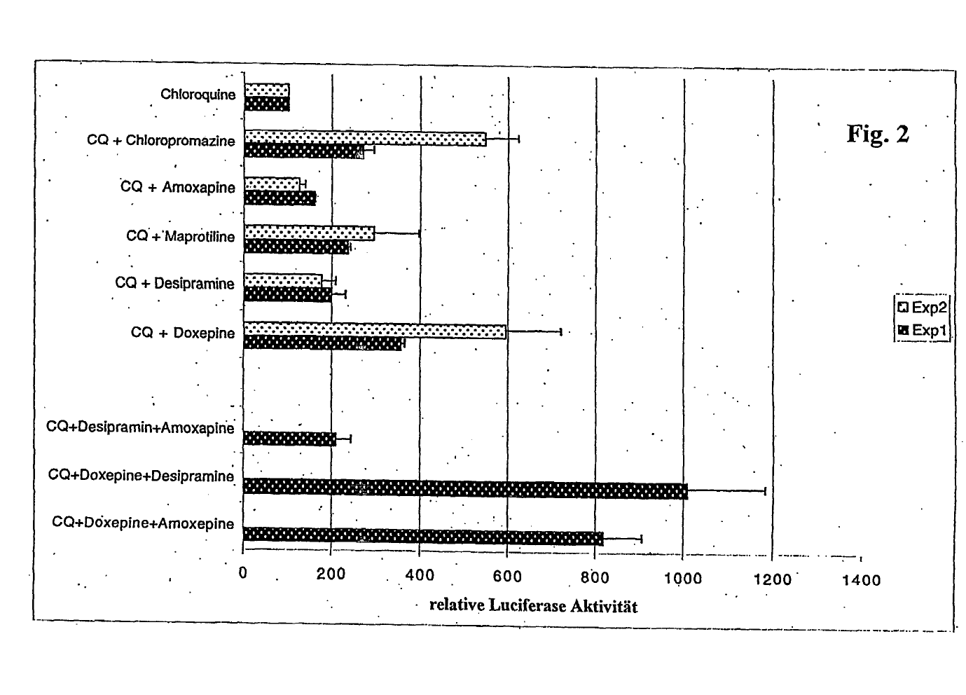

In FIG. 2, experiments with mixtures of substances used according to the invention are shown. Herein, the respectively optimum concentrations were used, as determined in example 3. CQ is chloroquine. In the case of mixtures of chloroquine with substances according to the invention, a continuously drastically increased luciferase activity is found, compared to the use of chloroquine alone. A comparison with FIG. 1b shows that even with regard to the use of the substances according to the invention alone, drastically increased values are obtained. In total, the use of such mixtures thus leads to a substantial improvement of the transfection efficiency, and thereby generally results a substantial improvement for promoting the endocytosis.

### EXAMPLE 5

### Dosages

Irrespective of the above embodiments, typically usable dosages of the tricyclic antidepressants used according to the invention are in the range of 1 to 200 mg per day (for an adult with 75 kg body weight), preferably 30 to 120 mg. The amount of the active ingredient in a composition according to the invention depends on the dosages being usual for the active ingredient. Preferably, the dosages of the active ingredient are equal to or smaller than the dosages, which are usual without administration of the ones used according to the invention. The dosages may be reduced by up to 90% and more.

1. The use of a substance according to formula I or II or of several different such substances 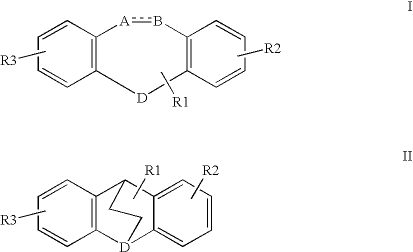
wherein A, B and D are identical or different and are each a C, N, S or O atom,
wherein . . . is a single or double bond,
wherein free valences are bound with —H,
wherein R1 is present one, two or three, in the case of formula II up to four times and is —H, —C1-15-alkyl (branched, linear or cyclic, saturated or unsaturated) or —C1-15-alkyl (branched, linear or cyclic, saturated or unsaturated) wherein one or several C atoms is substituted with by O or N,
wherein R1 in the case that A, B and/or D in formula I is a C atom, may be bound by a single or double bond to such a C atom,
wherein R2 and R3 are identical or different, each and independently from each other are present one, two, three or four times and are —H or -Hal, wherein -Hal=—F, —Cl or —Br,
for preparing a pharmaceutical composition comprising an active ingredient being different from the substance or the substances and having a molecular weight of larger than 300.

2. The use according to claim 1, wherein R1 is a secondary or tertiary amine.

3. The use according to claim 1 or 2, wherein R1 is present once and is bound to D, wherein in the case of formula II D is a C atom.

4. The use according to one of claims 1 to 3, wherein . . . is a single bond.

5. The use according to one of claims 1 to 4, wherein A and B are C atoms, and D is a C or N atom.

6. The use according to one of claims 1 to 4, wherein A is a C atom, B is an O atom, and D is a C atom.

7. The use according to one of claims 1 to 6, wherein R2 and R3 are —H.

8. The use according to one of claims 1 to 3, wherein A is an N atom, B is a C atom, and D is an O atom, and wherein . . . is a double bond.

9. The use according to claim 9, wherein R2 is present once and -Hal, and wherein R1 is —H.

10. The use according to one of claims 1 to 9, wherein the pharmaceutical composition in addition comprises a quinoline ring compound and/or a phenothiazine compound promoting the endocytosis or several different such compounds, optionally selected from the group consisting of “chloroquine, promazine, quinine, biquinoline, phenothiazine and chlorpromazine”.

11. The use according to one of claims 1 to 10, wherein the pharmaceutical composition contains the active ingredient, in particular a construct including a nucleic acid having a defined sequence or consisting thereof, in a mixture or in a separate packing unit.

12. The use of a substance according to one of claims 1 to 9 or of several different such substances, optionally in a mixture with a quinoline ring compound and/or a phenothiazine compound promoting the endocytosis, optionally selected from the group consisting of “chloroquine, primazine, quinine, biquinoline, phenothiazine and chlorpromazine”, for promoting the endocytosis of an active ingredient, in particular of a construct comprising a nucleic acid having a defined sequence or consisting thereof, wherein the cell is incubated in vitro with the active ingredient and the substance.

13. A method for transiently or stably transfecting a cell in vitro, wherein the cell is incubated with a construct comprising a nucleic acid having a defined sequence or consisting thereof, and a substance according to one of claims 1 to 9 or several different such substances, optionally in a mixture with a quinoline ring compound and/or a phenothiazine compound promoting the endocytosis, optionally selected from the group consisting of “chloroquine, primazine, quinine, biquinoline, phenothiazine and chlorpromazine”, wherein the substance causes the endocytotic of the nucleic acid, and wherein the nucleic acid transfects the cell.

14. A method according to claim 13, wherein the construct comprises a marker gene, or wherein the incubation is performed with a second construct including a nucleic acid coding for a marker gene or consisting thereof.

15. A stably or transiently transformed cell, obtained by that a cell is incubated with a construct comprising a nucleic acid having a defined sequence or consisting thereof, and a substance according to one of claims 1 to 9 or several different such substances, optionally in a mixture with a quinoline ring compound and/or a phenothiazine compound promoting the endocytosis, optionally selected from the group consisting of “chloroquine, primazine, quinine, biquinoline, phenothiazine and chlorpromazine”, wherein the substance causes the endocytosis of the construct, and wherein the nucleic acid transforms the cell.

16. The use of a mixture of at least two different substances selected from the group consisting of “quinoline ring compounds and/or a phenothiazine compounds promoting the endocytosis, alkyl polyamines, and the substance promoting the endocytosis according to one of claims 1 to 9” for preparing a pharmaceutical composition comprising an active ingredient different from the substances and having a molecular weight of larger than 300.
